SEQUENCE LISTING (3 sequences)

SEQ ID NO: 1 - protein, 185 aa, Bordetella pertussis
  PEPTIDE (1)..(185)
TVGHGDPHQGVLAQGDIIMDAKGGTLLLRNDALTENGTVTISADSAVLEHSTIESKISQSVLAAKGDKGKPAVSVKVAKKLFLNGTLRAVNDNNETMSGRQIDVVDGRPQITDAVTGEARKDESVVSDAALVADGGPIVVEAGELVSHAGGIGNGRNKENGASVTVRTTGNLVNKGYISAGKQGV

SEQ ID NO: 2 - protein, 4 aa, Unknown
  This signal peptide exists in many species for protein dimer formation.
GSGC

SEQ ID NO: 3 - protein, 4 aa, Unknown
  SIGNAL (1)..(4): This signal peptide exists in many eukaryotes as an endoplasmic reticulum retention signal.
HDEL

(19) United States
(12) Patent Application Publication
(10) Pub. No.: US 2008/0305124 A1
(43) Pub. Date: Dec. 11, 2008

(54) ORAL PERTUSSIS VACCINE AND METHOD FOR PRODUCING PERTUSSIS VACCINE

(75) Inventors: Shih-Tong Jeng, Taipei City (TW); Bor-Luen Chiang, Taipei City (TW); Meng-Huei Chang, Nantou City (TW); Hsi-Mei Lai, TAIPEI CITY (TW)

Correspondence Address: WPAT, PC;INTELLECTUAL PROPERTY ATTORNEYS, 2030 MAIN STREET, SUITE 1300, IRVINE, CA, 92614, US

(73) Assignee: AGRICULTURE AND FOOD AGENCY, COUNCIL OF AGRICULTURE, EXECUTIVE YUAN, Nantou County (TW)

(21) Appl. No.: 12/046,420

(22) Filed: Mar. 11, 2008

(30) Foreign Application Priority Data
Mar. 14, 2007 (TW) 096108745

(51) Int. Cl. A61K 39/10; A01H 5/00; C12N 15/82; C07K 14/415

(52) U.S. Cl. 424/190.1; 800/301; 800/279; 530/350

(57) ABSTRACT

The present invention is related to an oral pertussis vaccine and a method for producing pertussis vaccine, wherein the oral pertussis vaccine comprises amino acid sequence of 1094 to 1279 (SEQ ID NO.1) of filamentous hemagglutinin. The method for producing pertussis vaccine comprises following steps: constructing a vector comprising amino acid sequence of 1094 to 1279 (SEQ ID NO. 1) of filamentous hemagglutinin; transforming the vector into Agrobacterium successfully; co-culturing vector containing Agrobacterium with plant; and obtaining successfully a transformed plant as the vaccine. The present invention is also related to a polypeptide containing SEQ ID NO.1.

### FIELD OF THE INVENTION

This invention is related to an oral pertussis vaccine and a method for producing pertussis vaccine.

### BACKGROUND OF THE INVENTION

Pertussis is a contagious disease by contact of nose or throat discharges of infected individuals, and almost all non-immunized children and infants can be infected (Cherry et al., 1988, Report of the task force on pertussis and pertussis immunization. Pediatrics 81: 933-984). Pertussis, also known as whooping cough, gets its name from the whooping sound of patients due to serious cough. Studies have shown that filamentous hemagglutinin (FHA) of pertussis pathogen Bordetella pertussis is one of the virulent factors enabling attachment of pertussis pathogen to filament cells of respiratory system (Locht et al., 1989, Identification of amino acid residues essential for the enzymatic activities of pertussis toxin. Proc. Natl. Acad. Sci. USA 86: 3075-3079) for further damage through colony formation and toxin release.

Most of the conventional vaccines were whole-cell vaccines which have less efficacy and many side effects as their major drawbacks (Guzamn et al., 1991, Antibody responses in the lungs of mice following oral immunization with Salmonella typhimurium aroA and invasive Escherichia coli Strains expressing the filamentous hemagglutinin of Bordetella pertussis. Infect. Immun. 59: 4391-4397; Walker et al., 1992, Specific lung mucosal and systemic immune responses after oral immunization of mice with Salmonella typhimurium aroA, Salmonella typhi Ty21a, and invasive Escherichia coli expressing recombinant pertussis toxin S1 subunit. Infect. Immun. 60: 4260-4268).

Revealed patents of pertussis are listed below: Taiwan patent No. 190505 is related to an immunological antagonist using endotoxin from Bordetella pertussis as antigenic determinant; Taiwan patent No. 170603 is related to an immunological antagonist using filamentous hemagglutinin or the carbohydrate-containing antigen which can partially antagonize the immunoactivity of filamentous hemagglutinin; Taiwan patent No. 174730 is related to a vaccine which includes the FHA from Bordetella pertussis, detoxified LPF and a 69 kDa protein from cell outer membrane; Taiwan patent No. 149549 is related to a purification method of Bordetella pertussis antigen; EP0336736 is related to a purification method of Bordetella pertussis antigen; GB568384 is related to a method of producing Bordetella pertussis endotoxin and the product; JP60226822 is related to a purification method of filamentous hemagglutinin; CA1341123 is related to a genetically engineered full-length filamentous hemagglutinin as a vaccine. In current papers and journal articles, Journal of Microbiology Immunology and Infection 34(4) December 2001, 243-251 used purified filamentous hemagglutinin and pertussis toxin for immunization; Vaccine 18 (2000) 860-867 was using partial filamentous hemagglutinin, which was different from the present invension, as research target; FEMS Immunology and Medical Microbiology 23 (1999) 235-24b used a different 220 KD fragment from N terminus of filamentous hemagglutinin as antigenic determinant which was different from the present invention; Infection & Immunity 59(12) 1991. 4391-4397 used oral immunization with full-length filamentous hemagglutinin; and Infection & Immunity 59(9) 1991. 3313-3315 indicated that either N or C terminus of filamentous hemagglutinin can be used as antigen.

Most of the current vaccine developing systems use antigen as vaccine which can induce animal immune system via injection or oral medication to generate antibodies for further disease prevention.

Existing pertussis vaccine is injected into animal to induce immunization. Since pertussis infects through respiratory mucus, oral immunization via gastrointestinal tract mucus can induce more significant reaction than injection (Arakawa et al., 1997, Expression of cholera toxin B subunit oligomers in transgenic potato plants. Transgenic Res. 6: 403-413).

### SUMMARY OF THE INVENTION

The present invention provides an oral pertussis vaccine and a method for producing pertussis vaccine.

### BRIEF DESCRIPTION OF THE DRAWINGS

FIG. 1 shows the gel electrophoresis of Western blot analysis of the purified peptide with amino acid sequence of 1094 to 1279 of filamentous hemagglutinin from E. coli (lane 1, positive control), total proteins from either wild-type tobacco (lane 2, negative control) or various transgenic lines of tobacco (lanes 3-5).

FIG. 2 illustrates a bar graph of filamentous hemagglutinin recognizing IgG content analysis from rat blood by enzyme linked immunosorbent assay (ELISA). Purified filamentous hemagglutinin FAD from E. coli (E), total protein from wild-type tobacco (WT), and total protein from transgenic tobacco (FADT) were used to feed mice. After 35 days of feeding, mice serum samples were analyzed by ELISA to determine the amount of FAD specific IgG.

FIG. 3 illustrates the bar graph of filamentous hemagglutinin recognizing IgA content analysis from stool by enzyme linked immunosorbent assay (ELISA). Purified filamentous hemagglutinin FAD from E. coli (E), total protein from wild-type tobacco (WT), and total protein from transgenic tobacco (FADT) were used to feed mice. After 35 days of feeding, mice serum samples were analyzed by ELISA to determine the amount of FAD specific IgA.

### DETAILED DESCRIPTION OF THE INVENTION

The present invention relates to transforming the amino acid sequence of 1094 to 1279 of filamentous hemagglutinin of pertussis pathogen into plants by genetic engineering. In a preferred embodiment of the present invention, it provides edible plants for human consumption to prevent diseases caused by pertussis pathogen.

Accordingly, the present invention provides an oral pertussis vaccine which comprises amino acid sequence of 1094 to 1279 of filamentous hemagglutinin (Fragment A, FA, SEQ ID NO.1).

In a preferred embodiment, the amino acid sequence of 1094 to 1279 of filamentous hemagglutinin is transformed into an organism by genetic engineering, which enables the organism to possess amino acid sequence of 1094 to 1279 of filamentous hemagglutinin (SEQ ID NO. 1).

In a preferred embodiment, the organism is a plant and the vaccine comprises extract of the plant.

In a more preferred embodiment, the plant is an edible plant and the vaccine comprises extract of the edible plant.

The said oral pertussis vaccine of the present invention can further comprise a signal sequence of Gly-Ser-Gly-Cys (SEQ ID NO.2) which is operably linked to SEQ ID NO.1.

In a preferred embodiment, the amino acid sequence containing SEQ ID NO.1 and SEQ ID NO.2 is transformed into an organism by genetic engineering, which enables the organism to possess amino acid sequence containing SEQ ID NO.1 and SEQ ID NO.2.

The additional GSGC peptide (SEQ ID NO.2) helps Fragment A (SEQ ID NO.1) to form dimmer structure and thus enhance the protein's ability of inducing antibody production.

In a preferred embodiment, the organism is a plant and the vaccine comprises extract of the plant.

In a more preferred embodiment, the plant is an edible plant and the vaccine comprises extract of the edible plant.

The said oral pertussis vaccine of the present invention can further comprise a signal sequence of His-Asp-Glu-Leu (SEQ ID NO.3), which is operably linked to SEQ ID NO.1.

In a preferred embodiment, the amino acid sequence containing SEQ ID NO.1 and SEQ ID NO.3 is transformed into an organism by genetic engineering, which enables the organism to possess the amino acid sequence containing SEQ ID NO.1 and SEQ ID NO.3.

This additional HDEL peptide (SEQ ID NO.3) retains Fragment A (SEQ ID NO.1) in the Endoplasmic Reticulum of the cell of the transformed organism and prevents protein degradation in the Cytoplasm. The approach increases the Fragment A quantity contained in the transformed organism such as plant.

In a preferred embodiment, the organism is a plant and the vaccine comprises extract of the plant.

In a more preferred embodiment, the plant is an edible plant and the vaccine comprises extract of the edible plant.

The said oral pertussis vaccine comprising SEQ ID NO.1 and SEQ ID NO.2 can further comprise SEQ ID NO.3.

In a preferred embodiment, the amino acid sequence containing SEQ ID NO.1, SEQ ID NO.2 and SEQ ID NO.3 is transformed into an organism by genetic engineering, which enables the organism to possess the amino acid sequence containing SEQ ID NO.1, SEQ ID NO.2 and SEQ ID NO.3.

In a preferred embodiment, the organism is a plant and the vaccine comprises extract of the plant.

In a more preferred embodiment, the plant is an edible plant and the vaccine comprises extract of the edible plant.

The present invention also provides a method of producing pertussis vaccine, comprising:
a) constructing a vector comprising DNA encoding amino acid sequence of 1094 to 1279 (SEQ ID NO.1) of filamentous hemagglutinin from pertussis pathogen;
b) transforming the successfully constructed vector from step a) into an Agrobacterium;
c) co-culturing the vector containing Agrobacterium from step b) and a plant; and
d) obtaining the successfully transformed plant.

In a preferred embodiment, the successfully transformed plant is further extracted.

In a preferred embodiment, the said vector of the step (a) can further comprises a signal sequence of Gly-Ser-Gly-Cys (GSGC, SEQ ID NO.2) operably linked to SEQ ID NO.1.

In a preferred embodiment, the said vector of the step (a) can further comprise a signal sequence of His-Asp-Glu-Leu (HDEL, SEQ ID NO.3) operably linked to SEQ ID NO.1.

In a preferred embodiment, the said vactor of the step (a) comprising SEQ ID NO.1 and SEQ ID NO.2 can further comprises SEQ ID NO. 3 operably linked to SEQ ID NO.1 and SEQ ID NO.2.

In a preferred embodiment, the plant is an edible plant and the vaccine comprises extract of the edible plant.

The present invention also provides a polypeptide containing amino acid sequence of 1094 to 1279 (SEQ ID NO.1) of filamentous hemagglutinin and a signal sequence of Gly-Ser-Gly-Cys (SEQ ID NO.2) or His-Asp-Glu-Leu (SEQ ID NO.3) operably linked to SEQ ID NO.1.

In one embodiment, the polypeptide containing amino acid sequence of SEQ ID NO.1 and SEQ ID NO.2 can further contain SEQ ID NO.3.

The term “signal sequence” used herein indicates a sequence with specific physiological function on one end of a protein. In the present invention, the signal sequence is especially used to trigger the formation of protein dimmer structure or retain the protein in the Endoplasmic Reticulum.

The term “edible plant” used herein comprises edible crops and food crops, such as tomato, vegetable, papaya, banana, rice, tobacco etc.

The term “dimer structure” used herein indicates that the protein is formed by two monomers. In the present invention, dimer structure can significantly induce antibody production.

In the present invention, the amino acid sequence comprises 1094 to 1279 (SEQ ID NO. 1) of pertussis filamentous hemagglutinin with or without signal sequences (GSGC and/or HDEL) is used for plant transformation by genetic engineering technology. The transformed plant is for human consumption as well as for pertussis prevention.

Features and advantages of the present invention are revealed in the following examples.

### EXAMPLES

The following examples illustrate the present inventions as material and method guidance but not limited to the same.

### Example 1

### Plasmid Construction

The amino acid sequence of 1094 to 1279 of filamentous hemagglutinin in pUC13 was used for plasmid construction as Fragment A (FA) as shown in SEQ ID NO. 1:

TVGHGDPHQGVLAQGDIIMDAKGGTLLLRNDALTENGTVTISADSAVLEH
STIESKISQSVLAAKGDKGKPAVSVKVAKKLFLNGTLRAVNDNNETMSGR
QIDVVDGRPQITDAVTGEARKDESVVSDAALVADGGPIVVEAGELVSHAG
GIGNGRNKENGASVTVRTTGNLVNKGYISAGKQGV.

In order to form dimer structure of FA protein in plant cells to enhance the protein's ability of inducing antibody production, the signal sequence for protein dimer formation was constructed to the end of FA. Filamentous hemagglutinin (FHA) was used as template, and after polymerase chain reaction (PCR), the additional GSGC amino acid sequence for dimer formation was created at the end of FA. The PCR product, FAD, was cloned into pBI221 via BamHI and SacI sites. The clone was confirmed by DNA sequencing.

In order to direct FAD protein to retain in endoplasmic reticulum of plant cells, the signal sequence for endoplasmic reticulum retention was constructed to the end of FAD which already contained GSGC pepetide signal. FAD was used as template, and after polymerase chain reaction (PCR), the additional HDEL amino acid for endoplasmic reticulum retention was created at the end of FAD. The PCR product, FADH, was cloned into pCAMBIA2300 via EcoRI and HindIII sites. The clone was confirmed by DNA sequencing.

### Example 2

### Preparation of Agrobacterium Competent Cell

Picked up Agrobacterium LBA4404 signal colony and cultured it in 5 ml of LB for 2 days at 28° C. Subcultured 200 μl of culture into fresh 5 ml of LB medium for another 2 days at 28° C. Subcultured 200 μl of culture into fresh 5 ml of LB and distributed to multiple tubes depending on the number of transformation reaction required and cultured for 18 to 20 hours at 28° C. Kept bacteria culture on ice for 10 minutes, transferred 1.5 ml of bacteria into microcentrifuge tubes and then spun it at 4500 g for 5 minutes under 4° C. Removed supernatant, resuspended pellet with cold sterile water and then spun it at 4500 g for 5 minutes under 4° C. and removed supernatant. Repeated the washing step four times and then resuspended pellet in 40 μl of 10% glycerol for immediate transformation or further −70° C. storage after quick-freezing with liquid nitrogen.

### Example 3

### Agrobacterium Transformation

Mixed one tube of Agrobacterium competent cell with dialyzed DNA solution thoroughly and transferred the mixture into a 2 mm cuvette for electroporation with Electro Cell Manipulator ECM 600 Electroporation System (BTX) at 1.44 kV/129Ω. Immediately resuspended bacteria with 1 ml of LB. Cultured it at room temperature for 1 hour without shaking and then plated it on LB plates containing appropriate antibiotics. Cultured it at 28° C. and selected colonies after 2 days.

### Example 4

### Tobacco Transformation

Picked up Agrobacterium single colony containing desired gene for expression and cultured it in 5 ml of LB containing 100 mg/l kanamycin and 100 μM acetosyringone at 28° C. for 2 days. Cut leaves from tobacco tissue culture seedlings into small pieces and placed it on MS medium [MS salts (Murashige and Skoog, 1965), 0.4 mg/l BAP, 3% sucrose, 0.8% agar, pH 5.7]. Evenly distributed 100 μl of bacteria culture on the MS medium with tobacco leaves and incubated it at 27° C. under 16 hr light/8 hr dark (16L/8D) cycle for co-cultivation for 2 days. Then transfer tissue culture material to medium containing 200 mg/l kanamycin and 300 mg/l claforan at 27° C. under 16L/8D cycle for 3 weeks for top bud formation. Cut off the top bud and transferred it into RM medium (MS salt, 1.5% sucrose, 0.8% agar, pH 5.7) containing 100 mg/l kanamycin and 200 mg/l claforan. Roots started to develop in 1-2 weeks, and after that, it could grow into a whole plant.

### Example 5

### Antibody Preparation

Polyclonal anti-FHA antibody was separated from serum of BALA/c mice immunized with pertussis antigen FHA (Skelton and Wong, 1990) via intraperitoneal injection. Rabbit anti-mouse total IgG was purchased from Jackson Immuno Research Laboratories, Inc. Goat anti-mouse IgA was purchased from Southern Biotechnology Associates, Inc.

### Example 6

### Crude Extraction of Plant Protein

The appropriate amount of leaves was put in a mortar and grinded with liquid nitrogen. The powder was transferred into a centrifuge tube and the same volume of protein extraction buffer (10 mM sodium phosphate pH 7.3, 40 mM sodium ascorbate, 20 mM EDTA, 0.05% Tween-20) was added. The centrifuge tube was cooled down in ice until the powder dissolved, then well mixing ground tissue and extraction buffer by vortex. Spun it at 10,000 g for 15 minutes at 4° C. and saved the supernatant. Repeated the centrifugation step until supernatant was clear. The clear supernatant was the protein crude extract.

### Example 7

### Mice Feeding

B6 mice were purchased from Breeding Division of Laboratory Animal Center of National Taiwan University College of Medicine. Eight-week-old mice were used in feeding experiment. Mice were fed totally five times at day 0, 7, 14, 21 and 28. There were 3 subgroups with 5 mice in each group. 15 μg of filamentous hemagglutinin purified from E. coli, 20 mg total protein from wild-type tobacco, and 20 mg of total protein from tobacco transformed with filamentous hemagglutinin were used to feed each subgroup respectively. Each mouse was weighed before feeding for further body weight observation. Serum and stool samples were collected after 35 days of feeding.

### Example 8

### Sample Collection

### 8.1 Serum Sample Collection

Mice blood was collected by orbital breeding after the 35th days of feeding. Transferred blood into microcentrifuge tube and sat it at room temperature for 10 minutes. Spun it at 5,000 g for 10 minutes at 4° C. Transferred supernatant into a new microcentrifuge tube and spun it at 5,000 g for 5 minutes at 4° C. The supernatant was serum and was stored at −70° C. for further analysis.

### 8.2 Feces Sample Collection

After 35 days of feeding, mice of the same subgroup were placed in a cabinet for 1 hour before collecting all the feces samples. Distributed 0.2 g of feces sample in each microcentrifuge tube and stored it at −70° C. for further analysis. Added 0.4 ml of PBS (10 mM sodium phosphate, 150 mM NaCl pH 7.3) into each tube, vortex vigorously, sat for 15 minutes at room temperature and then spun it at 12,000 g for 15 minutes at 4° C. The supernatant was stored at −70° C. for further analysis.

### Example 9

### ELISA (Enzyme-Linked Immunosorbent Assay)

Antigen was diluted with 50 mM NaHCO₃ (pH 9.6) to reach the final concentration of 0.5 μg/ml. Added 100 μl of diluted antigen into each well on ELISA plate, and then sat it at 4° C. for overnight. Removed solution from well, washed it with PBST (10 mM sodium phosphate, 150 mM NaCl, 0.05% Tweeen-20 pH 7.3) for 3-5 times. Added 200 μl of 1% BSA-PBS (1% bovine serum albumin, 10 mM sodium phosphate, 150 mM NaCl pH 7.3) as blocking solution, and incubated it at 37° C. for 2 hours. Removed solution from wells, washed it with PBST for 4 times, added 100 μl of serum or feces sample diluted with 1% BSA-PBS and incubated it at 37° C. for 3 hours. Removed solution from wells, washed it with PBST for 5 times, added 100 μl of secondary antibody with horseradish peroxidase conjugate in 1000 fold dilution with 1% BSA-PBS, and sat it at 37° C. for 2 hours. Removed solution from wells, washed it with PBST for 5 times, added 100 μl of ABTS [0.1 M citrate-phosphate, 0.01% H2O2, 0.6 mg/ml ABTS (2,2′ azino-bis (3-ethylbenzothiazoline-6-sulfonic acid) diammonium salt)] for 5 minutes and then detected OD₄₂₀ with Spectra Max plus Microplate Spectrophotometer.

### Example 10

### Results

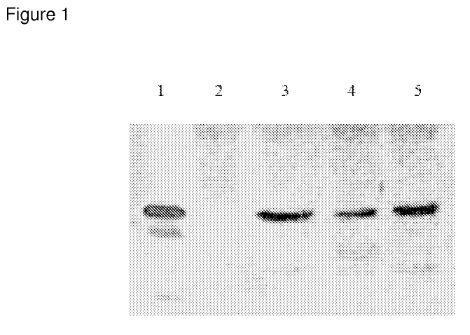

Purified fragment of filamentous hemagglutinin from E. coli, total proteins from either wild-type tobacco or various transgenic lines of tobacco were analyzed by Western blot analysis. The result demonstrated that transgenic tobacco containing partial filamentous hemagglutinin gene could express fragment of filamentous hemagglutinin containing amino acid sequence of 1094 to 1279. As shown in FIG. 1, lane 1 indicates the peptide with amino acid sequence of 1094 to 1279 of filamentous hemagglutinin purified from E. coli as a positive control, lane 2 indicates total protein purified from wild-type tobacco as a negative control, and lanes 3-5 indicates total protein purified from various transgenic tobacco lines.

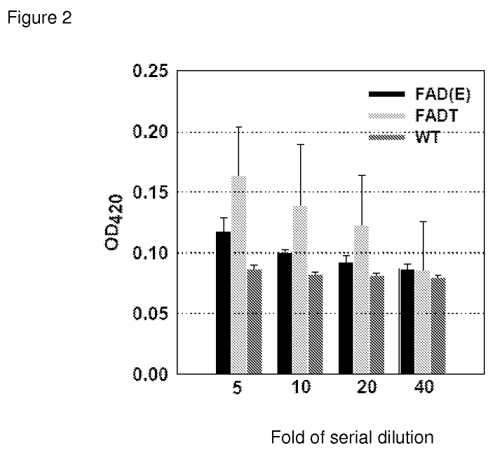

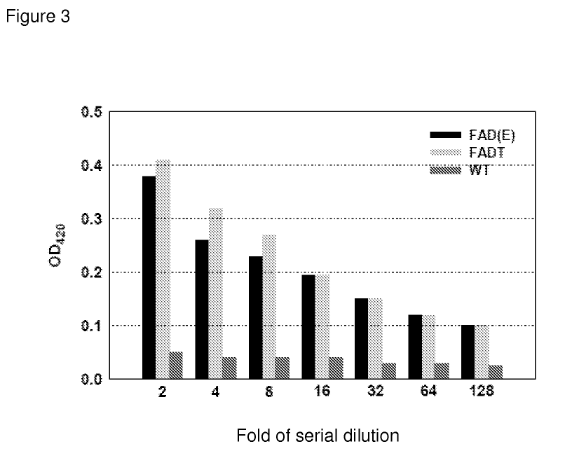

Furthermore, purified filamentous hemagglutinin FAD from E. coli (E), total protein from wild-type tobacco (WT), and total protein from transgenic tobacco (FADT) were used to feed mice. After 35 days of feeding, mice serum samples were analyzed by ELISA to determine the amount of FAD specific IgG. The amount of FAD specific IgA from stool samples was also determined. As shown in FIG. 2 and FIG. 3, the average and standard deviation were determined from analysis with more than five mice. The result shown that mice fed with transgenic tobacco total protein could successfully induce pertussis antigen specific antibody in mice.

Based on the present invention, various modification and changes can be made without deviation of the scope and focus of the present invention. The present invention described specific preferred embodiment, however, the present invention should not be limited to the preferred embodiments. In terms of listed preferred embodiments, obvious modifications are also included in claims to the person skilled in the art.

What is claimed is:

1. An oral pertussis vaccine which comprises amino acid sequence of SEQ ID NO.1.

2. The vaccine of claim 1, wherein said amino acid sequence of SEQ ID NO. 1 is transformed into an organism by genetic engineering, which enables the organism to possess the amino acid sequence of SEQ ID NO. 1.

3. The vaccine of claim 2, wherein the said organism is an edible plant.

4. The vaccine of claim 3, which comprises extract of the edible plant.

5. The vaccine of claim 1, which further comprises a signal sequence of SEQ ID NO. 2 operably linked to SEQ ID NO.1.

6. The vaccine of claim 5, wherein said amino acid sequence containing SEQ ID NO.1 and SEQ ID NO.2 is transformed into an organism by genetic engineering, which enables the organism to possess the amino acid sequence containing SEQ ID NO.1 and SEQ ID NO.2.

7. The vaccine of claim 6, wherein the said organism is an edible plant.

8. The vaccine of claim 7, which comprises extract of the edible plant.

9. The vaccine of claim 1, which further comprises a signal sequence of SEQ ID NO.3 operably linked to SEQ ID NO.1.

10. The vaccine of claim 9, wherein said amino acid sequence containing SEQ ID NO.1 and SEQ ID NO.3 is transformed into an organism by genetic engineering, which enables the organism to possess the amino acid sequence containing SEQ ID NO.1 and SEQ ID NO.3.

11. The vaccine of claim 10, wherein the said organism is an edible plant.

12. The vaccine of claim 11, which comprises extract of the edible plant.

13. The vaccine of claim 5, which further comprises a signal sequence of SEQ ID NO.3 operably linked to SEQ ID NO.1 and SEQ ID NO.2.

14. The vaccine of claim 13, wherein said amino acid sequence containing SEQ ID NO.1, SEQ ID NO.2 and SEQ ID NO.3 is transformed into an organism by genetic engineering, which enables the organism to possess the amino acid sequence containing SEQ ID NO.1, SEQ ID NO.2 and SEQ ID NO.3.

15. The vaccine of claim 14, wherein the said organism is an edible plant.

16. The vaccine of claim 15, which comprises extract of the edible plant.

17. A method of producing pertussis vaccine, comprising:
a) constructing a vector comprising DNA encoding amino acid sequence of SEQ ID NO.1;
b) transforming the successfully constructed vector from step a) into an Agrobacterium;
c) co-culturing the transformed Agrobacterium from step b) and a plant; and
d) obtaining the successfully transformed plant.

18. The method of claim 17, which further comprises extracting the successfully transformed plant.

19. The method of claim 17, wherein the vector of the step (a) further comprises a signal sequence of SEQ ID NO.2 operably linked to SEQ ID NO. 1.

20. The method of claim 17, wherein the vector of the step (a) further comprises a signal sequence of SEQ ID NO.3 operably linked to SEQ ID NO.1.

21. The method of claim 19, wherein the vector of the step (a) further comprises a signal sequence of SEQ ID NO.3 operably linked to SEQ ID NO.1 and SEQ ID NO.2.

22. The method of claim 17, wherein the said plant is an edible plant.

23. A polypeptide containing SEQ ID NO.1 and SEQ ID NO.2 operably linked together.

24. The polypeptide of claim 23, which further contains SEQ ID NO.3 operably linked to SEQ ID NO.1 and SEQ ID NO.2.

25. A polypeptide containing SEQ ID NO.1 and SEQ ID NO.3 operably linked together.
